(19) United States
(12) Patent Application Publication
(10) Pub. No.: US 2011/0184499 A1
(43) Pub. Date: Jul. 28, 2011

(54) SKIN TREATMENT DEVICE AND SYSTEM

(76) Inventor: Robert Radi, La Quinta, CA (US)

(21) Appl. No.: 13/015,494

(22) Filed: Jan. 27, 2011

Related U.S. Application Data
(60) Provisional application No. 61/298,822, filed on Jan. 27, 2010.

(51) Int. Cl. A61N 5/06; A61N 7/00

(52) U.S. Cl. 607/88; 601/2

(57) ABSTRACT

The present invention provides, in at least one embodiment, a skin care device and system for treating skin. In an exemplary embodiment of the invention, a skin care device implements a high-frequency sonic or ultrasound emitter to exfoliate, cleanse, and smooth skin in combination with light therapy. Advanced microfiber brush heads are interchangeable and vary in color to provide different colored light therapy to the skin. The present invention can be used every day to help refresh skin cells, improve surface texture, and prepare the skin to better absorb cosmeceutical beauty treatments.

### CROSS REFERENCE TO RELATED APPLICATIONS

This application claims priority under 35 U.S.C. §119(e) to U.S. Provisional Application No. 61/298,822, filed Jan. 27, 2010, and entitled “SONICFLOW SKIN TREATMENT DEVICE AND SYSTEM,” the disclosure of which is hereby incorporated by reference in its entirety.

### BACKGROUND OF THE INVENTION

1. Field of Invention

The invention relates generally to skin treatment and therapy, and more particularly, to a device and system for cleansing and improving the health of treated skin through ultrasound, light therapy, and/or cosmeceuticals.

2. Description of Related Art

Skin care is related to enhancing a skin's appearance, cleanliness, and health, and can be accomplished by soap, damage preventative measures, skin-care creams, suntan lotion, moisturizing lotions, powders, makeup, baby products, bath oils, bubble baths, bath salts, butters, and many other types of products. Skin care products are often formulated for different skin types, such as sunscreens to protect the skin from UV radiation and damage, skin lighteners, and treatment products that repair or hide skin imperfections (e.g., acne, wrinkles, dark circles under eyes, etc.), and tanning oils to brown the skin.

Ultrasound is cyclic sound pressure with a frequency greater than the upper limit of human hearing (about 20 KHz). The production of ultrasound is used in many different fields, typically to penetrate a medium and measure the reflection signature or supply focused energy. The reflection signature can reveal details about the inner structure of the medium. Ultrasound is used in many applications, though the most well-known application is to produce pictures of fetuses in the human womb.

Light therapy consists of exposure to daylight or to specific wavelengths of light using lasers, light-emitting diodes, fluorescent lamps, dichroic lamps or very bright, full-spectrum light, usually controlled with various devices. The light is administered for a prescribed amount of time and, in some cases, at a specific time of day. Light therapy has been used in treatment of the skin such as acne, in treatment of sleep disorders by putting light in the retina, and in treatment of some psychiatric disorders such as seasonal affective disorder (i.e., winter depression).

U.S. Pat. No. 7,066,941 describes an apparatus for skin treatment, where aging or damaged skin is treated by irradiating affected skin areas with an effective amount of visible light emanating from an optical apparatus having a wavelength of about 400 nm to about 500 nm. Green light (about 500 to about 590 nm) may be used as adjunct therapy with blue/violet light in some embodiments.

U.S. Pat. No. 5,843,072 describes a method and device for treatment of unwanted veins by a combined sclerotherapy and light treatment. Near infra-red wavelength light is preferably used in the light therapy due to its better depths of penetration through the skin to deeper lying, larger vessels such as varicose veins.

U.S. Pat. No. 5,259,380 describes a light therapy system. With a designated wave length in the infrared bandwidth and 5-10 minute treatment regimens of continuous and pulsed operations has been effective in alleviating various musculoskeletal disorders, skin ulcers, and delayed post-operative wound healing.

Conventional methods fall short on providing an ultrasonic and light therapy method to exfoliate. cleanse, and smooth skin.

### SUMMARY OF THE INVENTION

The present invention provides a skin care device and system for treating skin. In an exemplary embodiment of the invention, a skin care device implements a high-frequency sonic or ultrasound emitter to exfoliate, cleanse, and smooth skin in combination with light therapy, Advanced microfiber brush heads are interchangeable and vary in color to provide variations in the colored light therapy to the skin, or the light is translucent to enhance the light therapy onto the skin.

In an embodiment of the invention, a device comprises: an ultrasound source for emitting ultrasound; and a colored or translucent detachable brush, wherein the emitted ultrasound is delivered through the colored or translucent detachable brush. The colored or translucent detachable brush may emit red light, blue light, or a mixture of the two lights within the device. The device may further comprise a light switch, the light switch switching light between blue light, red light, and the mixture of red light and blue light. The device may further comprise a charging station, a power switch, and a low level laser.

In another embodiment of the invention, a device comprises: a translucent brush; a light source within the brush, the light source configured to provide red or blue light or a mixture of the two within the device; and a vibration source, the vibration source configured to transmit ultrasound frequencies. The ultrasound frequency may be greater than 20 Hz or 20 KHz. The brush may comprise bristles and receive a cosmeceutical product. The device may further comprise a charging station and a power switch.

In another embodiment of the invention, a method of treating skin comprises steps of: applying ultrasound to a skin treatment area; and applying colored light to the skin treatment area. The colored light may be red or blue light or a mixture of the two. The steps may further comprise applying a cosmeceutical to the skin treatment area.

Advantages of the present invention include better refreshing of skin cells, improved surface texture and elasticity, and better preparation the skin to better absorb cosmeceutical beauty treatments though cleansing and exfoliation.

The foregoing, and other features and advantages of the invention, will be apparent from the following, more particular description of the preferred embodiments of the invention, the accompanying drawings, and the claims.

### BRIEF DESCRIPTION OF THE DRAWINGS

For a more complete understanding of the present invention, and the advantages thereof, reference is now made to the ensuing descriptions taken in connection with the accompanying drawings briefly described as follows:

FIG. 1 illustrates a skin treatment device according to an embodiment of the invention;

FIG. 2 illustrates the skin treatment device of FIG. 1 with a red colored detachable brush or a clear translucent brush lit by a red light source;

FIG. 3 illustrates the skin treatment device of FIG. 1 with a blue colored detachable brush or a clear translucent brush lit by a blue light source; and

FIG. 4 illustrates the skin treatment device of FIG. 1 with a charging station according to an embodiment of the invention.

### DETAILED DESCRIPTION OF EMBODIMENTS

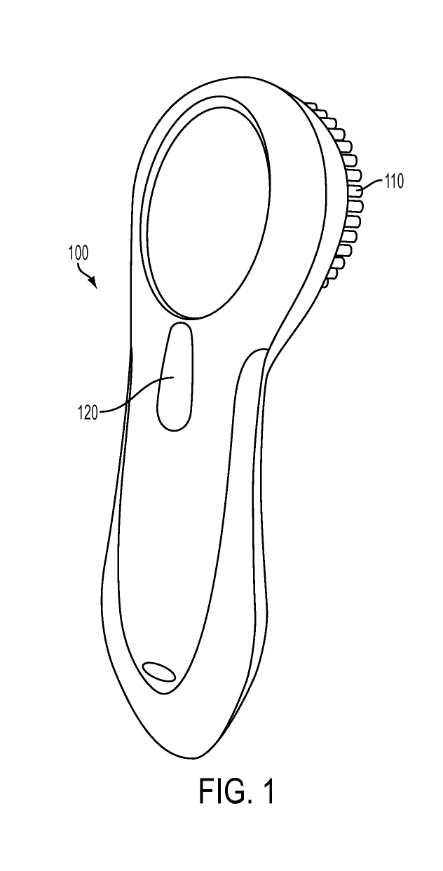

Further features and advantages of the invention, as well as the structure and operation of various embodiments of the invention, are described in detail below with reference to the accompanying FIGS. 1-4, wherein like reference numerals refer to like elements. Although the invention is described in the context of a hand-held cordless form factor, the device can take any form-factor capable of delivering ultrasound and light radiation to a treatment area of a user's skin.

The present invention provides a skin care device and system for treating skin. In an exemplary embodiment of the invention, a skin care device implements a high-frequency sonic or ultrasound emitter to exfoliate, cleanse and smooth skin in combination with light therapy. The device provides a professional rejuvenating treatment at home. The device can be used to help refresh skin cells, improve surface texture, and prepare skin to better absorb special cosmeceutical beauty treatments. The device provides gentle micropulsating vibrations to reveal smooth, healthy skin. Advanced microfiber brush heads and two power settings make the device suitable for all skin types, even delicate or sensitive skin. The brush heads are interchangeable and vary in color to enhance the delivery of different colored light therapy to the skin. Each brush attachment features a different color, namely blue, red, and clear translucent, in order to enhance the delivery of different colors of light to the skin treatment area. The present invention can be used every day to help refresh skin cells, improve surface texture and prepare the skin to better absorb cosmeceutical beauty treatments.

FIG. 1 illustrates a skin treatment device 100 according to an embodiment of the invention. The skin treatment device 100 is a lightweight hand-held device for treating skin and comprises a detachable brush 110 and a power switch 120. The power switch 120 may also include a multi-speed switch in order to permit the user to select different vibration or ultrasound frequencies. As shown, the brush 110 is a white or clear colored brush, which may be transparent or semi-transparent, for delivering light emitted from a light source (not shown) within the device 100 to a skin treatment area. The device 100 further comprises a pressure source (not shown), the implementation of which is apparent to one of ordinary skill in the art, for producing and delivering cyclic sound pressure, such as ultrasound, through the brush 110. In an exemplary embodiment of the invention, the ultrasound has a frequency around 20 KHz, although any ultrasound frequency down to 20 Hz may be implemented. The detachable brush 110 may be interchanged with different colored brushes as noted below. Thus, the device 100 implements sonic technology to exfoliate, cleanse, and smooth skin in combination with light therapy.

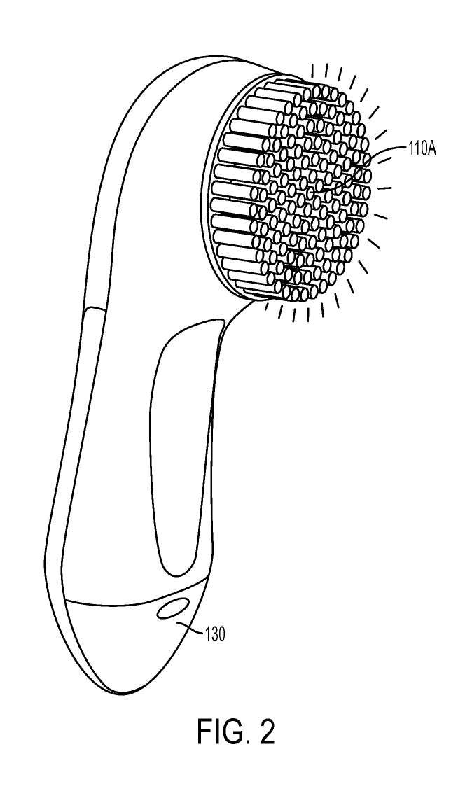

FIG. 2 illustrates the skin treatment device 100 with a red colored detachable brush or clear translucent 110A. The detachable brush 110A is translucent to allow light to flow through its bristles. The light flowing through the bristles is filtered so that only red light is emitted due to the red nature of the brush 110A. In one embodiment, red light includes light 660 nm out of UV range. For example, the base of the brush 110A may be a translucent shade of red to enhance the red light reaching the skin. The bristles may be clear or red as well. Red light is known to, among other things, stimulate skin tissue, reducing lines and wrinkles, promote collagen production, and improving skin firmness to achieve anti-aging benefits. The device 100 further comprises a rechargeable battery housing 130 for housing rechargeable batteries, the identification and implementation of which are apparent to one of ordinary skill in the art. In an alternative embodiment of the invention, the batteries may be standard (not rechargeable) batteries. In yet another embodiment of the invention, the device 100 implements a power adapter for powering the device 100 from an electrical source such as a wall electrical socket.

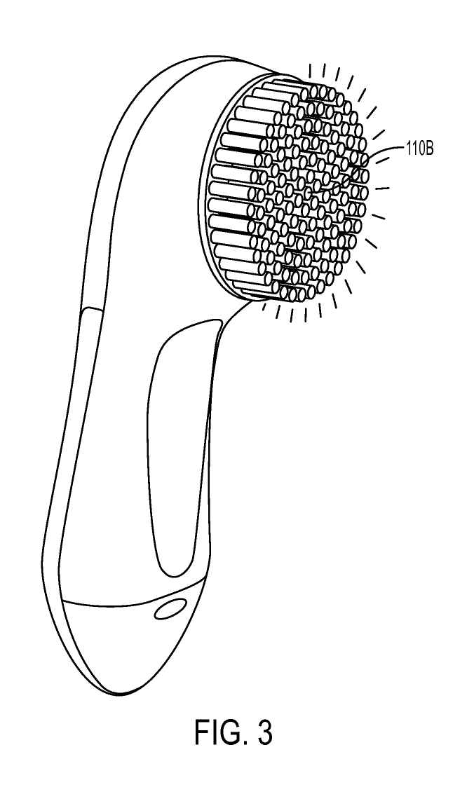

FIG. 3 illustrates the skin treatment device 100 with a blue colored detachable brush or clear translucent 110B. The detachable brush 110B permits blue light to reach skin through its bristles. In one embodiment, blue light includes light 415 nm out of UV range. For example, the base of the brush 110B may be a translucent shade of blue to enhance the blue light reaching the skin. The bristles may be clear or blue as well. Blue light is known to, among other things, kill bacteria and reduce acne blemishes.

In one embodiment, the device 100 has a light switch (not shown) that switches the light between blue light, red light, and one or more mixtures of blue and red light. In an alternative embodiment of the invention, the skin treatment device 100 includes red (or infrared) and blue (or ultraviolet) sources of tight, and the detachable brush 110 is clear to transmit such light to the skin.

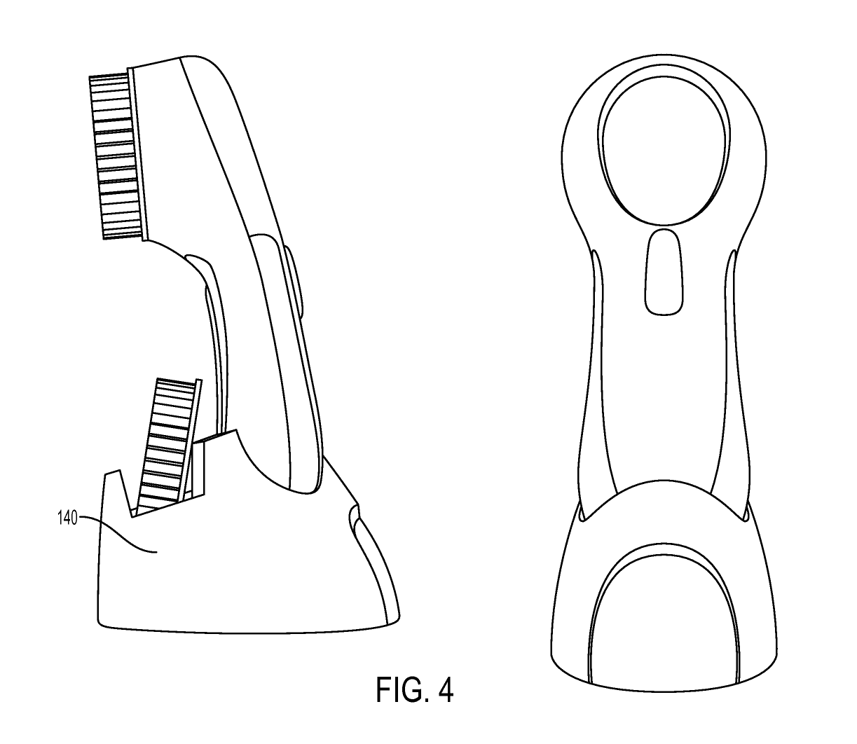

FIG. 4 illustrates the skin treatment device 100 with a charging station 140 according to an embodiment of the invention. The charging station 140 and batteries of the device are inductively coupled, the implementation of which is apparent to one of ordinary skill in the art, in order to wirelessly recharge the batteries of the device. The charging station 140 may further include a recess as shown for storing one or more of the detachable brushes 110.

In an embodiment of the invention, the device 100 may further include an internal motor (not shown) for mechanically moving, e.g., rotating, the bristles of the brush 110. For example, the brush 110 may move in micro-orbits, i.e., circular vibrations. In an exemplary embodiment, the circular vibrations occur at a rate of 5,600 vibrations per minute to a maximum of 18,000 vibrations per minute.

In an embodiment of the invention, the device 100 may further include a low level laser, also known as a cold laser, low power laser, soft laser, biostimulation laser, therapeutic laser, laser acupuncture, etc. The low level laser can be above the light source of the brush 110, alters cellular function, and is effective for skin related treatment such as Anti-Aging Light Therapy, Fine Lines & Wrinkles, Collagen Stimulation, Broken Capillaries, Acne/Blackhead & Whitehead Removal, Rosacea/Couperose, Psoriasis, Sun Damaged Skin Cellulite. Body Firming.

The device 100 can be used with exemplary cosmeceuticals such as, but not limited to, a misty cleanser, a rejuvenating treatment, a balancing toner, and a repair facial mask. Cosmeceuticals represent the marriage of cosmetics and pharmaceuticals. They are intended to enhance the health and beauty of the skin by altering its structure. Cosmeceutical products contain bioactive ingredients including vitamins, enzymes, antioxidants, ceramides, minerals, herbs and essential oils. These skin care products go beyond coloring and adorning the skin, as they can actually improve the functioning of the skin, encourage collagen growth, and make the skin healthier. Cosmeceuticals may be helpful in preventing and diminishing the signs of premature aging, wrinkles, fine lines, and age spots. Certain key ingredients are essential for changes in the skin to take place. These ingredients may be found in small amounts in cosmetics. Cosmeceuticals, however, contain larger percentages of these ingredients, boosting their ability to effect changes in the skin. Examples of products typically labeled as cosmeceuticals include anti-aging creams and moisturizers. Cosmeceuticals are cosmetic products with biologically active ingredients that have medical or drug-like benefits. The device 100 may be operated after applying a cosmeceutical to either the brush 110 or the area of the skin being treated. Operation of the device 100 enhances the absorption of the cosmeceutical into the skin.

It is to be recognized that depending on the embodiment, certain acts or events of any of the methods described herein can be performed in a different sequence, may be added, merged, or left out altogether (for example, not all described acts or events are necessary for the practice of the method). Moreover, in certain embodiments, acts or events may. be performed concurrently, rather than sequentially.

The invention has been described herein using specific embodiments for the purposes of illustration only. It will be readily apparent to one of ordinary skill in the art, however, that the principles of the invention can be embodied in other ways. Therefore, the invention should not be regarded as being limited in scope to the specific embodiments disclosed herein, but instead as being fully commensurate in scope with the following claims.

I claim:

1. A device comprising:
an ultrasound source for emitting ultrasound; and
a colored or clear translucent detachable brush, wherein the emitted ultrasound is delivered through the colored or clear translucent detachable brush.

2. The device of claim 1, wherein the colored or clear translucent detachable brush emits red light within the device.

3. The device of claim 1, wherein he colored or clear translucent detachable brush emits blue light within the device.

4. The device of claim 1, wherein the colored or clear translucent detachable brush emits a mixture of red light and blue light within the device.

5. The device of claim 4, further comprising a light switch, the light switch switching light between blue light, red light, and the mixture of red light and blue light.

6. The device of claim 1, further comprising a charging station.

7. The device of claim 1, further comprising a power switch.

8. The device of claim 1, further comprising a low level laser.

9. A skin treatment device comprising:
a translucent brush;
a light source within the brush. the light source configured to provide red light, blue light, or a mixture of red and light light within the device; and
a vibration source, the vibration source configured to transmit ultrasound frequencies.

10. The device of claim 9, wherein the ultrasound frequency is greater than 20 Hz.

11. The device of claim 9, wherein the ultrasound frequency is greater than 20 KHz.

12. The device of claim 9, wherein the brush comprises bristles.

13. The device of claim 9, wherein the brush receives a cosmeceutical product.

14. The device of claim 9, further comprising a charging station.

15. The device of claim 9, further comprising a power switch.

16. The device of claim 9, further comprising a low level laser.

17. A method of treating skin, the method comprising the steps of:
applying ultrasound to a skin treatment area; and
applying colored light to the skin treatment area.

18. The method of claim 17, wherein the colored light is red light.

19. The method of claim 17, wherein the colored light is blue light.

20. The method of claim 17, wherein the colored light is a mixture or red and blue light.
